(19) United States
(12) Patent Application Publication
(10) Pub. No.: US 2005/0038318 A1
(43) Pub. Date: Feb. 17, 2005

(54) Gastrointestinal tool over guidewire

(76) Inventor: Benad Goldwasser, Tel Aviv (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/639,442

(22) Filed: Aug. 13, 2003

(51) Int. Cl. A61B 1/00

(52) U.S. Cl. 600/104; 600/109; 600/114; 604/93.01

(57) ABSTRACT

Gastrointestinal apparatus including a guidewire, and a gastrointestinal tool formed with a bore, the guidewire passing through the bore.

### FIELD OF THE INVENTION

This invention relates generally to gastrointestinal tools, such as endoscopes and colonoscopes, and more particularly to gastrointestinal tools that slide over a guidewire, guiding catheter and the like, and which may be introduced into and through the colon, e.g., to the cecum, by any means.

### BACKGROUND OF THE INVENTION

Endoscopy has become an increasingly important tool in diagnosing and in treating ailments of the gastrointestinal tract, also referred to as the GI tract. Typical endoscopes are essentially formed by a somewhat flexible tube that is pushed through the GI tract, after being introduced in the body cavity starting from the rectum or starting from the esophagus. The endoscope has a steerable tip to facilitate navigation through the GI tract, and typically has to be sufficiently stiff so that it can be pushed further along the body cavity. The tip of the endoscope that is introduced in the GI tract can be outfitted with several devices, most notably an illumination device and a vision device, such as a vision integrated circuit, so that the operator of the endoscope can observe the interior of the GI tract and maneuver the endoscope in the proper position.

Once the endoscope is in position, other tools attached to the endoscope or inserted through the endoscope can be brought to the proper position in the GI tract. Various procedures can then be carried out, such as removing polyps, performing sutures, irrigation, suction, and removing other tissues. The various tools that are used together with the endoscope can be either inserted separately in the GI tract and placed in the proper position independently, or may travel in a working channel of the endoscope, so that once the endoscope is positioned at the desired location in the GI tract, the tools inserted in the endoscope will also easily reach that position.

Endoscopes or other smaller similar devices can also be used to explore other body cavities, for example airways, genitourinary tract, female reproductive organs, etc., or blood vessels. These probes must be small to fit in the smaller cavities, and care must be taken to avoid damage to the more fragile membranes lining these cavities.

Current state of the art endoscopes are very capable devices, and endoscopy has been very successful in diagnostic and therapeutic applications with the use of current endoscopes and the current arsenal of tools that can be inserted through the working channel of the endoscope, or can be attached to the outside of the endoscope. However, current endoscope technology has limitations and drawbacks. One of the greatest drawbacks of current endoscopes is that the working channel is small. The working channel is small relative to overall diameter of the endoscope, and is further limited by the space taken up by vision, irrigation, suction, light, and control cabling mechanisms that are part of the endoscope and are required to control the endoscope. Thus there is a very small area left for other tools to be introduced through the endoscope. Also, the additional channels may make passage of the endoscope through body cavities more difficult, as they contribute among other things to its diameter.

U.S. Pat. No. 6,517,477 to Wendlandt, assigned to Scimed Life Systems, Inc. (Maple Grove, Minn., U.S.), describes a catheter introducer system for endoscopy that includes a steering section and a propulsion section located near the end of the flexible, tubular catheter that is introduced in a body cavity. The propulsion section is designed to pull the rest of the catheter inside the body cavity, so there is no need to push the catheter along from outside the body. Propulsion may be accomplished by relatively movable gripping pads that selectively apply suction to the tissue. The steering section is designed to point the end of the catheter that is introduced into the body cavity in the desired direction. The catheter may be made very flexible in bending, and a larger diameter catheter may be used.

### SUMMARY OF THE INVENTION

The present invention seeks to provide an improved gastrointestinal endoscope with tools that may be slid over a guidewire, as is described in detail hereinbelow.

It is noted that the term “guidewire” as used throughout the specification and claims, encompasses any instrument which may be introduced into a body lumen, such as but not limited to, a guiding catheter, monorail, wire, hollow or not hollow, with or without segments, and of any material property (e.g., flexibility).

There is thus provided in accordance with an embodiment of the present invention gastrointestinal apparatus comprising a guidewire, and a gastrointestinal tool formed with a bore, the guidewire passing through the bore.

In accordance with an embodiment of the present invention the guidewire comprises a distal stop that prevents movement of the gastrointestinal tool therepast.

Further in accordance with an embodiment of the present invention the gastrointestinal tool comprises an imaging device, such as but not limited to, a CCD camera, an illumination device, a vision device, an ultrasound sensor, and/or an x-ray emitter. Additionally or alternatively, the gastrointestinal tool may comprise a cutting tool, a sampling device and/or a magnetic device.

Still further in accordance with an embodiment of the present invention the gastrointestinal tool may comprise a catheter that slides over the guidewire. The catheter may comprise a lumen for passing therethrough at least one of an instrument and a fluid.

In accordance with an embodiment of the present invention the gastrointestinal tool comprises a chamfer for facilitating passage through a lumen.

There is also provided in accordance with an embodiment of the present invention a method for constructing a gastrointestinal apparatus, comprising providing a guidewire, providing a gastrointestinal tool formed with a bore, and sliding the gastrointestinal tool over the guidewire, the guidewire passing through the bore. In addition, another tool may be slid over the guidewire that cooperates with the gastrointestinal tool. As another alternative, a collapsible sleeve may be slid over the guidewire and inflated to functionally create an endoscope with single or multiple channels that run through its length.

### BRIEF DESCRIPTION OF THE DRAWINGS

The present invention will be understood and appreciated more fully from the following detailed description taken in conjunction with the appended drawings in which:

FIG. 1 is a simplified illustration of a guidewire, in accordance with an embodiment of the present invention, introduced through the rectum into the large intestine all the way to the cecum;

FIG. 2 is a simplified illustration of an imaging device and sleeve slid over the guidewire of FIG. 1, in accordance with an embodiment of the present invention; and

FIG. 3 is a simplified illustration of additional devices introduced over the guidewire of FIG. 1, such as but not limited to, a catheter with a lumen for passing therethrough wires (electrical or other) and another lumen for introducing therethrough fluids and the like, in accordance with an embodiment of the present invention.

### DETAILED DESCRIPTION OF PREFERRED EMBODIMENTS

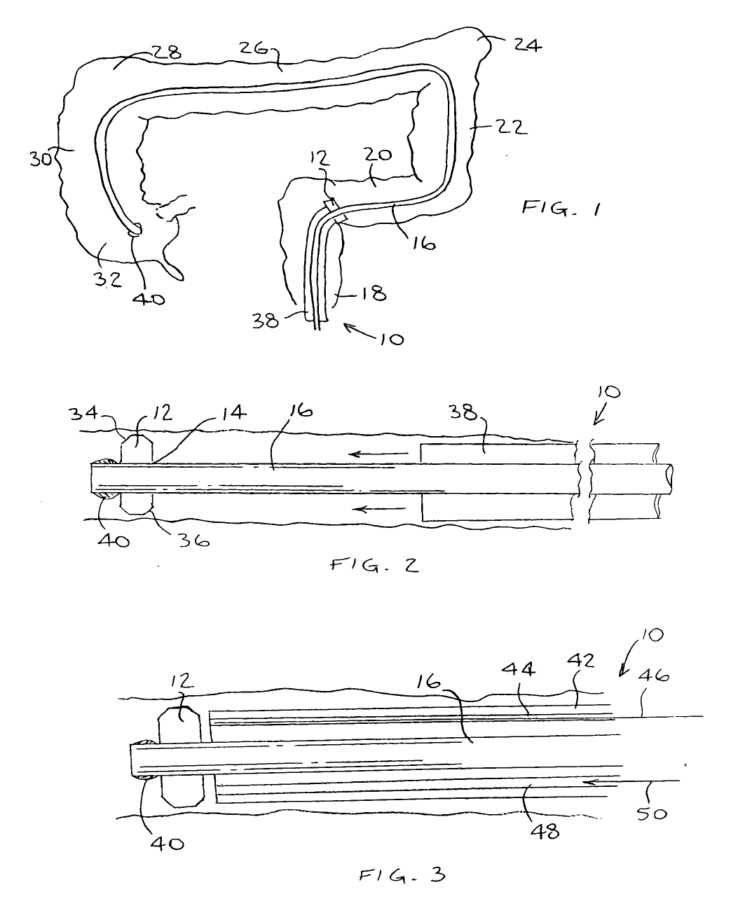

Reference is now made to FIGS. 1 and 2, which illustrate gastrointestinal apparatus 10, constructed and operative in accordance with an embodiment of the present invention.

Gastrointestinal apparatus 10 may comprise a gastrointestinal tool 12 formed with a bore 14 that slides over a guidewire 16. The guidewire 16 passes through bore 14. Examples of gastrointestinal tools 12 are described hereinbelow. Guidewire 16 may be constructed of any suitable flexible, medically safe material, such as a plastic or metal.

The following description follows for introducing guidewire 16 into the gastrointestinal tract, particularly the colon. However, it is emphasized that the invention is not limited to the GI tract and may be used for any other body lumen.

As seen in FIG. 1, guidewire 16 may be introduced through the rectum 18, past the sigmoid colon 20 and descending colon 22, where it may bend past the splenic flexure 24, pass through the transverse colon 26, bend past the hepatic flexure 28, pass into the ascending colon 30 and reach the cecum 32 by any method. For example, guidewire 16 may be pushed through the colon by hand as in typical colonoscopic procedures. Alternatively, guidewire 16 may be pulled through the colon, such as by a self-propelled mechanical device (or other devices, such as but not limited to, inflatable devices, electrical devices and the like). Gastrointestinal tool 12 may then be introduced into the colon by sliding over guidewire 16. Gastrointestinal tool 12 may be formed with a distal chamfer 34 to facilitate movement distally into the colon. Additionally, gastrointestinal tool 12 may be formed with a proximal chamfer 36 to facilitate extraction from the colon.

A pushing device 38, such as but not limited to, a sleeve or catheter, may be slid over guidewire 16 to distally push gastrointestinal tool 12 over guidewire 16. Guidewire 16 may comprise a distal stop 40 that prevents distal movement of gastrointestinal tool 12 therepast. Distal stop 40 may be inflatable, wherein inflation of distal stop 40 may be useful in fixing the position of guidewire 16 in the colon.

In accordance with an embodiment of the present invention, gastrointestinal tool 12 may comprise an imaging device. The imaging device may comprise, without limitation, a CCD camera, an illumination device, a vision device, an ultrasound sensor, and/or an x-ray emitter.

Reference is now made to FIG. 3. The gastrointestinal tool may further comprise a catheter 42 that slides over guidewire 16. Catheter 42 may comprise a lumen (also referred to as a channel) 44 for passing therethrough an instrument 46. Additionally or alternatively, catheter 42 may comprise a lumen (also referred to as a channel) 48 for passing therethrough a fluid 50. Catheter 42 may be rigid or flexible, and may have a one-piece or multiple-piece construction. Catheter 42 may comprise an inflatable, collapsible sleeve, as is described hereinbelow.

Catheter 42 may include any number of lumens for multiple applications. Catheter 42 may be constructed of any suitable medically safe material, such as a plastic or metal. Catheter 42 may have a generally fixed form, or may be made of a collapsible material (e.g., nylon), wherein after partial or complete introduction into the body lumen, catheter 42 may be inflated to functionally create an endoscope with single or multiple channels that run through its length.

In accordance with another embodiment of the present invention, instrument 46 or gastrointestinal tool 12 may comprise a cutting tool or a sampling device for performing a variety of medical procedures, such as but not limited to, removing polyps, performing sutures, irrigation, suction, and removing other tissues.

In accordance with an embodiment of the present invention, gastrointestinal tool 12 may comprise a magnetic device, such as for coupling with other GI devices. For example, the magnetic device may be used to magnetically attract magnetic boluses or other ingestible objects used for tracking or imaging the GI tract. Alternatively, gastrointestinal tool 12 may comprise any other suitable attachment or attraction device, such as but not limited to, adhesives or fasteners for attracting and affixing to such boluses or other ingestible objects.

In accordance with an embodiment of the present invention, substances may be introduced in the GI tract via gastrointestinal tool 12 and or guidewire 16. Such substances may include, without limitation, materials injected for image contrast or labeling, such as but not limited to, x-ray dyes, radioactivity-tagged materials or radiopharmaceuticals, magnetic resonance imaging (MRI) contrast agents, and others, or pharmaceuticals, relaxants, and other medicinal substances. As another example, a fluid (e.g., air) may be introduced in the GI tract via gastrointestinal tool 12 and or guidewire 16 for inflating the colon to enhance capturing images thereof.

The present invention thus provides a unique way of constructing a gastrointestinal apparatus by sliding gastrointestinal tool 12 over guidewire 16. Subsequently other tools may be slid over guidewire 16 to cooperate with gastrointestinal tool 12. As mentioned before, a collapsible sleeve (e.g., which may take the form of catheter 42 shown in FIG. 3) may be slid over guidewire 16 and inflated to functionally create an endoscope with single or multiple channels (e.g., 44 or 48) that run through its entire or partial length.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

1. Gastrointestinal apparatus comprising:
a guidewire; and
a gastrointestinal tool formed with a bore, said guidewire passing through said bore.

2. The gastrointestinal apparatus according to claim 1, wherein said guidewire comprises a distal stop that prevents movement of said gastrointestinal tool therepast.

3. The gastrointestinal apparatus according to claim 1, wherein said gastrointestinal tool comprises an imaging device.

4. The gastrointestinal apparatus according to claim 3, wherein said imaging device comprises at least one of a CCD camera, an illumination device, a vision device, an ultrasound sensor, and an x-ray emitter.

5. The gastrointestinal apparatus according to claim 1, wherein said gastrointestinal tool comprises a catheter that slides over said guidewire.

6. The gastrointestinal apparatus according to claim 5, wherein said catheter comprises a lumen for passing therethrough at least one of an instrument and a fluid.

7. The gastrointestinal apparatus according to claim 1, wherein said gastrointestinal tool comprises a chamfer for facilitating passage through a lumen.

8. The gastrointestinal apparatus according to claim 1, wherein said gastrointestinal tool comprises a cutting tool.

9. The gastrointestinal apparatus according to claim 1, wherein said gastrointestinal tool comprises a sampling device.

10. The gastrointestinal apparatus according to claim 1, wherein said gastrointestinal tool comprises a magnetic device.

11. A method for constructing a gastrointestinal apparatus, comprising:
providing a guidewire;
providing a gastrointestinal tool formed with a bore; and
sliding said gastrointestinal tool over said guidewire, said guidewire passing through said bore.

12. The method according to claim 11, further comprising sliding another tool over said guidewire that cooperates with said gastrointestinal tool.

13. The method according to claim 11, wherein said gastrointestinal tool comprises an imaging device.

14. The method according to claim 11, wherein said gastrointestinal tool comprises a catheter that slides over said guidewire.

15. The method according to claim 11, further comprising sliding a collapsible sleeve over said guidewire and inflating said sleeve to create an endoscope, said inflated sleeve comprising at least one channel.
